(19) United States
(12) Patent Application Publication
(10) Pub. No.: US 2013/0189687 A1
(43) Pub. Date: Jul. 25, 2013

(54) METHOD FOR MEASURING PYROPHOSPHORIC ACID AND SNP TYPING METHOD

(71) Applicant: PANASONIC CORPORATION, Osaka (JP)

(72) Inventor: Hiroyuki TANAKA, Kyoto (JP)

(73) Assignee: PANASONIC CORPORATION, Osaka (JP)

(21) Appl. No.: 13/832,826

(22) Filed: Mar. 15, 2013

Related U.S. Application Data
(63) Continuation of application No. PCT/JP2012/002763, filed on Apr. 20, 2012.

(30) Foreign Application Priority Data
Jun. 6, 2011 (JP) 2011-125971

(51) Int. Cl. C12Q 1/68; C12M 1/34

(52) U.S. Cl. 435/6.11; 435/287.2

(57) ABSTRACT

One general aspect provides a method for detecting pyrophosphoric acid in a sample solution with high sensitivity and high accuracy by a small sensor element, and an SNP typing method. In the general aspect, a sample solution having a volume of more than that of a measurement cavity is supplied to the measurement cavity through a flow path, so as to expose a droplet from the opening. The droplet has a shape of sphere. The shape of the sphere is maintained by surface tension generated on a surface of the droplet. At least part of the sample solution contained in the droplet is evaporated so as to increase a concentration of pyrophosphoric acid in the sample solution included in the measurement cavity.

### CROSS-REFERENCE TO RELATED APPLICATIONS

This is a continuation application of International application No. PCT/JP2012/002763, with an international filing date of Apr. 20, 2012, which claims priority to Japanese patent application No. JP 2011-125971 as filed on Jun. 6, 2011, the content of which is incorporated herein by reference.

### BACKGROUND OF THE INVENTION

1. Field of the Invention

The technical field relates to a method for measuring pyrophosphoric acid in a sample solution stably with high sensitivity by a small sensor, and an SNP typing method using the same.

2. Description of the Related Art

### Background Art

In recent years, the market for molecule diagnosis, which includes external diagnostic drugs, has been rapidly spreading, and techniques about genetic codes have actively developed. In the medical field, by analyzing genes related to a disease, treatment against the disease at a molecular level has been becoming possible. By diagnosis using genetic analysis, tailor-made medical treatments corresponding to individual patients have also been becoming possible. In tailor-made medical treatments, genetic codes of the respective patients are analyzed in constitution diagnoses or infectious-disease diagnoses before drug treatment, and appropriate treatments or medications are conducted for individual patients. Thus, in-situ diagnoses are desirable, and it has been desirable to develop a rapid and easy method having high POCT (point of care testing) property.

Among genetic codes, genetic polymorphisms are important. As people's faces, figures and others are various, people's genetic codes are also considerably different between individuals. Among these genetic code differences, differences about each of which a change in base sequences is present at a frequency of 1% or more of population are called genetic polymorphisms. It is said that these genetic polymorphisms are related to not only individuals' facial forms but also causes for various genetic diseases, and individuals' constitutions, drug-responsibilities, drug side-effects and others. At present, examinations have been made about relation between these genetic polymorphisms and diseases or others.

In recent years, out of these genetic polymorphisms, attention has been paid, for example, to an SNP (single nucleotide polymorphism). An SNP denotes such a genetic polymorphism that among base sequences for a genetic code, one base is different. It is said that 2,000,000 to 3,000,000 SNPs are present in human genome DNAs. Any SNP is easily used as a marker for a genetic polymorphism. For this reason, it is expected that SNPs are applied to clinical use. At present, as SNP-related techniques, researches have been made about the identification of the position of an SNP in the genome, relationship between the SNP and a disease, and others, and further developments have been made about an SNP typing technique for determining the base of SNP site.

As the SNP typing technique, there are techniques of various types, such as a technique using hybridization, a technique using a restriction enzyme, and a technique using ligase or a similar enzyme. Among these techniques, there is a technique that uses primer elongation reactions. In this technique, SNP typing is attained by determining whether a primer elongation reaction is generated or not. As a detection method based on the SNP typing technique using the primer elongation reaction, the following methods have been devised: a method of using a fluorescent dye to detect an actual amplification product of DNA, and a method of using an immobilized probe electrode to detect the product electrically. Besides these methods, a method of detecting pyrophosphoric acid, which is a side product of the synthesis of nucleic acid by DNA polymerase, has also been devised. In this method, in order to detect a difference among progresses of the elongation reactions, pyrophosphoric acid generated in accordance with the progress of the primer elongation reaction is converted to ATP, and thereafter a luciferase reaction is used to measure the amount of pyrophosphoric acid (see Non Patent Document 1).

On the other hand, an investigation has been made about a method of detecting specifically pyrophosphoric acid generated in the step of a DNA elongation reaction through an enzyme reaction. There is a method of causing ATP sulfurylase to act onto pyrophosphoric acid, and then generating light in a luciferase-luciferin reaction, thus detecting pyrophosphoric acid (Patent Document 1).

However, this method may be problematic at the present time since, based on current technologies, an apparatus therefor becomes large since light detection is made. Nevertheless, it is anticipated that future advances may reduce the size of the apparatus and alleviate such problems.

On the other hand, without using light detection, a method of detecting pyrophosphoric acid electrochemically has been investigated.

Patent Document 2 discloses a method of: supplying a sample to a reaction system containing a DNA probe having a sequence complementary to an SNP sequence of DNA and having an SNP site, DNA polymerase, and deoxynucleotide; elongating the DNA probe by a PCR reaction; converting pyrophosphoric acid produced in accordance with the elongation reaction of the DNA probe to inorganic phosphoric acid by pyrophosphatase; further using a measuring system containing glyceraldehyde-3-phosphate, oxidized nicotinamide adenine dinucleotide, glyceraldehyde3-phosphate dehydrogenase, diaphorase, and potassium ferricyanide as an electron mediator to measure the value of a current through electrodes finally, thus typing the SNP sequence of DNA. It is stated that according to this method, the SNP sequence can be determined within 100 seconds of a time when the sample containing pyrophosphoric acid is added to the measuring system.

In this method, the measurement of pyrophosphoric acid and the typing of an SNP can be attained by measuring the redox reaction of the electron mediator electrochemically. This method is disclosed as a high-sensitive and easy method without requiring any optical system (Patent Document 2).

Furthermore, it is disclosed that measurement can be made more sensitive by arranging buffer solution components, enzymes, and the others in a reaction system to be optimally separated from each other (Patent Document 3).

When these detecting methods are each used to perform SNP typing, a template of DNA is first extracted from, for example, a patient's blood as a specimen. In order to make, at this time, patient's physical and mental burdens small, for example, the amount of a specimen taken out from the patient may be made small. When diagnostic items are various, the respective typings of a plurality of kinds of SNPs are simultaneously performed; thus, a plurality of sensor elements are used. It is desirable to make, at this time, the amount of a specimen used in one of the sensor elements small. From this background, it is desirable to develop a small SNP typing sensor coping with a small amount of a sample solution.

### PRIOR ART DOCUMENTS

### Patent Documents

Patent Document 1: JP 2002-369698
Patent Document 2: WO 03/078655
Patent Document 3: Japanese Patent No. 4202407

### Non Patent Documents

Non Patent Document 1: J. Immunological Method, 156, 55-60, 1992
Non Patent Document 2: Science and Technology of Advanced Material, 6 (2005) 671

### SUMMARY

One non-limiting and exemplary embodiment has been made to solve the above-mentioned problems. The non-limiting and exemplary embodiment provides a detecting method and an SNP typing method for detecting pyrophosphoric acid in a sample solution with high sensitivity and high accuracy by a small sensor element.

In one general aspect, the techniques disclosed here feature a method for detecting pyrophosphoric acid in a sample solution with high sensitivity and high accuracy by a small sensor element, and an SNP typing method. In the general aspect, a sample solution having a volume of more than the volume of a measurement cavity, 17 is supplied to the measurement cavity 17 through a flow path 18 so as to expose a droplet 211 from the opening 110. The droplet 211 has a shape of sphere. The shape of the sphere is maintained by surface tension generated on a surface of the droplet 211. At least part of the sample solution contained in the droplet 211 is evaporated so as to increase a concentration of pyrophosphoric acid in the sample solution included in the measurement cavity 17.

According to the general aspect, it is possible to provide a detecting method and an SNP typing method for detecting pyrophosphoric acid in a sample solution with high sensitivity and high accuracy by a small sensor element.

Additional benefits and advantages of the disclosed embodiments will be apparent from the specification and Figures. The benefits and/or advantages may be individually provided by the various embodiments and features of the specification and drawings disclosure, and need not all be provided in order to obtain one or more of the same.

### BRIEF DESCRIPTION OF THE DRAWINGS

FIG. 1 is a perspective view illustrating a structure of an electrode substrate according to one embodiment.

FIGS. 2A to 2F are sectional views illustrating a method for measuring pyrophosphoric acid and an SNP typing method according to one embodiment.

FIG. 3A is a schematic view of a measuring system demonstrating a method for measuring pyrophosphoric acid and an SNP typing method according to one embodiment.

FIG. 3B is a measurement sequence demonstrating the method for measuring pyrophosphoric acid and an SNP typing method according to one embodiment.

FIG. 4 is a graph showing measurements of one embodiment.

FIG. 5 is a graph showing measurements of one embodiment.

FIG. 6 is a perspective view illustrating a structure of an electrode substrate of a conventional pyrophosphoric acid sensor.

FIGS. 7A and 7B are sectional views illustrating a structure of a conventional pyrophosphoric acid sensor when the sensor is made small.

FIGS. 8A to 8C are sectional views showing one example of a compatibility between a reduction in the size of a conventional electrochemical sensor and a rise in the sensitivity thereof.

FIGS. 9A to 9C are each photographs showing results of one embodiment of the present invention.

### DETAILED DESCRIPTION

Finding on which the present disclosure has been based The present inventors have made eager researches about the sensor described in the column “Background Art” to make investigations thereon repeatedly, and have gained the following finding.

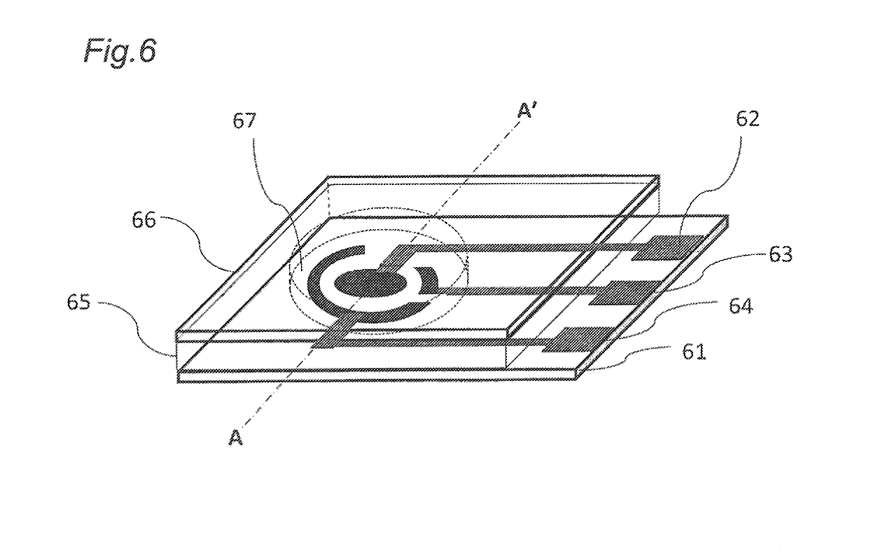

For example, in the measurement of pyrophosphoric acid and the typing of an SNP as illustrated in FIG. 6, a sensor element may be used in which a measurement electrode 62, a counter electrode 63 and a reference electrode 64 are formed on an insulating substrate 61 and a measurement cavity 67 is made. By filling a sample solution into the measurement cavity 67, and applying a constant voltage to the measurement electrode 62 to detect a current, pyrophosphoric acid can easily be measured. When a necessary reagent is dried and carried on the insulating substrate 61 of this sensor element, a labor or time for an advance preparation of the sample solution is saved, thus allowing the measurement to be made by an easy operation.

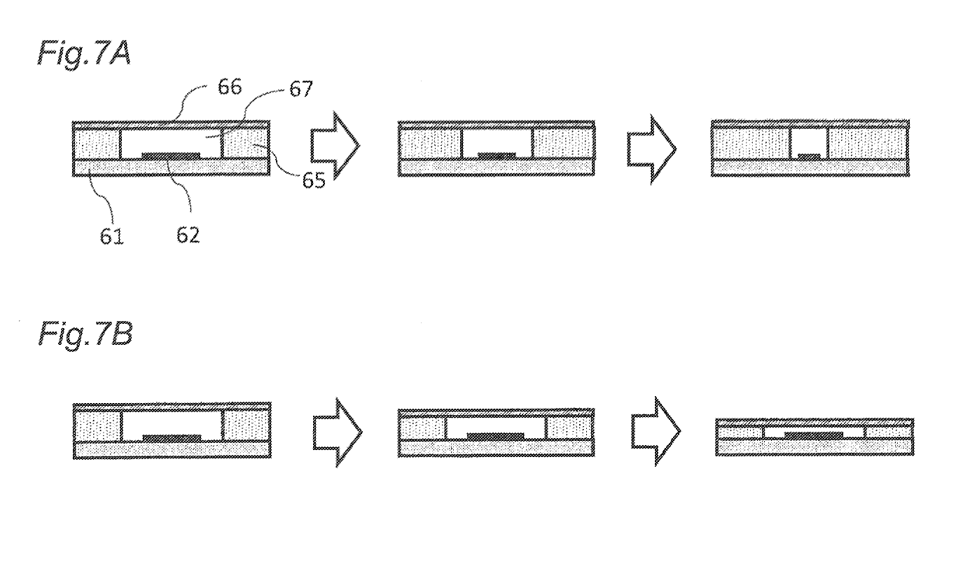

However, in cases where the amount of a sample solution containing a specimen is small and a sensor therefor becomes small, deterioration of the sensor in detecting sensitivity becomes a problem. Referring to FIG. 7, reason therefor is described below.

In order to cope with the sample solution in a small amount, for example, the diameter of a container of the sensor is made small while the height of the container is made constant (FIG. 7A). However, according to this method, it is indispensable to make the area of the measurement electrode smaller as the area of the bottom surface of the container is made smaller. Since the value of a detected current is in proportion to the electrode area, the decrease in the measurement electrode area results in a fall in the sensor sensitivity.

In order to cope with the sample solution in a small amount in another method, for example, the height of the container is made small while the electrode area is made constant (FIG. 7B). In this method, however, in a case where the height of the container is less than the thickness (about 300 μm or less) of a diffusion layer of reaction species involved in a current in the sample solution, the reaction species may be insufficiently supplied. Thus, during the measurement, the detected current value is apparently decreased. The decrease in the detected current value results in a fall in the sensor sensitivity. Furthermore, this method also has a defect that sensors cannot be arranged at a high density since the arrangement cannot ensure superiority in area.

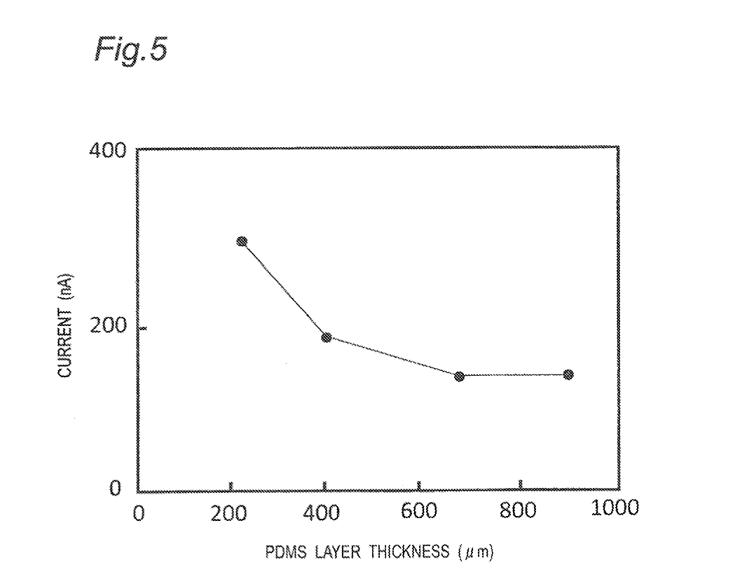

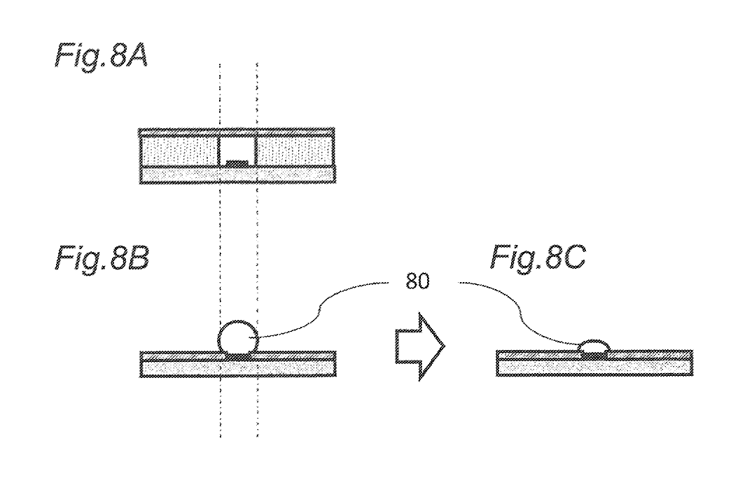

A method has been hitherto suggested for solving such problems, which evaporates part of a solvent component in a sample solution intentionally to raise the concentration of an electrochemical reaction species in the specimen, and then makes a measurement (for example Non Patent Document 2). According to this method, compared with a method using a system in which a solvent in a sample solution is not evaporated with the same measurement electrode area used (FIGS. 8A and 8B), a droplet 80 of the sample solution is concentrated by the evaporation (FIG. 5C) so that the amount of the reaction species reaching the electrode per unit area increases and the detected current value is raised. In other words, the concentration by the evaporation compensates for a fall in the detection sensitivity caused by a reduction in electrode area, thus resulting in the raised detection sensitivity. However, according to the disclosed method, the droplet 80 is always exposed to the atmosphere throughout the measurement. Accordingly, the degree of the concentration is easily affected by the external temperature or humidity, resulting in a problem of deteriorating the accuracy of the measurement. Particularly, when the amount of the sample solution is small, the speed that the concentration advances tends to increase Thus, it is difficult to control the timing of the measurement, so that the measurement accuracy reduces. As a result, this method has not yet been put into practice.

That is, when the sensor is made small, a conflicting exists between the stabilization of the detected current value, and a rise in the sensitivity by increase of the detected current which is caused by the concentration of the reaction species involved in the reaction, following the evaporation of the sample solution. A difficulty of compatibility therebetween is conventionally a problematic.

Considering the above-mentioned point, the present inventors have found out a detecting method of pyrophosphoric acid and an SNP typing method for detecting pyrophosphoric acid in a sample solution with high sensitivity and high accuracy by a small sensor element.

One embodiment of the present disclosure relates to a method for detecting pyrophosphoric acid contained in a sample solution. The method according to the present embodiment for detecting pyrophosphoric acid contained in a sample solution includes:
(a) preparing a pyrophosphoric acid sensor including:
an insulating substrate;
a measurement electrode provided on the insulating substrate;
a counter electrode provided on the insulating substrate;
a measurement cavity side wall provided on the insulating substrate;
the measurement cavity side wall having a measurement cavity which is a through hole provided in the inside the measurement cavity side wall;
a measurement cavity lid which overlaps the measurement cavity when viewed from a normal direction of the insulating substrate; and
a flow path which communicates with the measurement cavity; wherein
when viewed from the normal direction of the insulating substrate, the measurement cavity overlaps a portion of the measurement electrode and a portion of the counter electrode;
the measurement cavity lid includes an opening which is a through hole,
when viewed from the normal direction of the insulating substrate, the opening is included in the measurement cavity;
(b) supplying the sample solution having a volume of more than the volume of the measurement cavity to the measurement cavity through the flow path, so as to expose a droplet from the opening; wherein
the droplet has a shape of sphere;
the shape of the sphere is maintained by surface tension generated on a surface of the droplet;
(c) evaporating at least part of the sample solution contained in the droplet, so as to increase a concentration of the pyrophosphoric acid in the sample solution included in the measurement cavity;
(d) measuring a current value flowing through the sample solution using the measurement electrode and the counter electrode; and
(e) determining the concentration of the pyrophosphoric acid on the basis of the current value measured in the measuring (d).

Hereinafter, embodiments of the present disclosure will be described with reference to the drawings.

### Embodiment 1

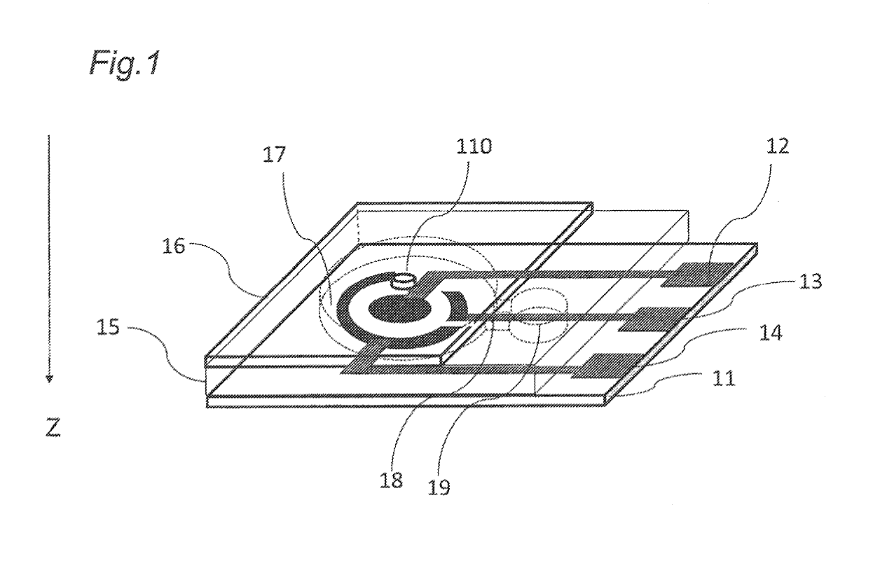

FIG. 1 is a perspective view illustrating a structure of an electrode substrate according to one embodiment of the present disclosure. A measurement electrode 12, a counter electrode 13 and a reference electrode 14 are formed on an insulating substrate 11. Each of the electrodes may be selected from a film of a noble metal such as gold, platinum or palladium, a carbon film, and others. Desirably, gold may be selected in view of, for example, the stability of the surface state.

The reference electrode 14 may be a reference electrode exhibiting non-polarization in view of the stability of electrical potentials thereof in a solution. A silver/silver chloride electrode may be selected because of easy handleability, and others. A method for forming the silver/silver chloride electrode may, for example, includes a method of depositing a silver plating or silver thin film onto a reference electrode moiety of an electrode traces made of, e.g., gold or platinum, and applying a voltage to the reference electrode in a an aqueous sodium chloride solution to cause the surface of the electrode to be silver chloride; a method of using a silver/silver chloride paste material to form an electrode body; a method of bringing an aqueous solution of, for example, sodium hypochlorite into contact with the surface of a silver paste; or the like.

Each of the electrodes is electrically connected through a conductive traces to a terminal part which is a part through which the electrode is connected to the external circuit. The conductive traces and the terminal part are desirably made of the same material as that used in the part of the electrode from the viewpoint of the production process. A method for forming the electrode and the conductive traces onto the insulating substrate 11 would be, for example, a method of sputtering or vapor-depositing a conductive material, and then trimming unnecessary portions thereof by etching using photolithography, or a laser; or direct sputtering for a traces of the electrode, using a stencil mask.

The insulating substrate 11 may be a substrate obtained by depositing an insulating thin film onto a supporting substrate of a semiconductor such as silicon. Alternatively, glass, ceramic material, resin or some other may be selected for the substrate 11. In order to obtain fine sensors once with good productivity, for example, an insulating substrate with compatibility with a semiconductor process may be used. For example, a substrate may be used which is obtained by forming a silicon oxide film or silicon nitride film into a thickness of 100 nm to 1 μm onto a silicon wafer by thermal CVD or plasma CVD.

The material of each of a measurement cavity side wall 15 and a measurement cavity lid 16 needs to be selected from materials unreactive with the sample liquid, and can be selected from semiconductors such as silicon and germanium, glasses such as quartz glass, lead glass and borosilicate glass, ceramic materials, resins, and others. Considering production easiness, use as a disposable biosensor, workability and costs, it is desirable to select a resin material. The resin material may be selected from polycarbonate (PC), polystyrene (PS), polypropylene (PP), polyimide (Pl), polytetrafluoroethylene (PTFE), polyphenylene sulfide (PPS), polyetheretheretherketone (PEEK), polyethylene terephthalate (PET), polymethyl methacrylate (PMMA), polyethylene-2,6-naphthalate (PEN), cyclic olefin copolymer (COC), polydimethylsiloxane (PDMS), and others. A method for forming the cavity side wall 15 and the cavity lid 16 may be, for example, cutting or etching of the substrate, molding using a mold, or embossing using thermal transfer.

For example, for the measurement cavity side wall 15, PDMS is used. Considering formation of a small sensor element, for the method for forming the measurement cavity side wall 15, a method capable of making a highly precise alignment with the electrode part easily may be used, and a method of using a transparent mold to transfer PDMS onto the substrate by a molding method may be used.

A surface of the measurement cavity lid 16 is, for example, subjected to a water repellency treatment with, for example, HMDS (hexamethyldisilane).

A measurement cavity 17 is connected to a filling opening 19 through a flow path 18. For example, the measurement cavity 17, the flow path 18 and the filling opening 19 are made of the same material as that used in the measurement cavity side wall 15 by the same method as that used for the measuring cavity side wall 15.

A method for forming an opening 110 in the measurement cavity lid 16 may be selected from mechanical cutting, cutting using a laser, etching, and other methods. For example, the opening 110 may be located just above the measurement electrode 12. The opening 110 desirably is circular. The diameter of the opening 110 is a diameter that permits at least substances contained in the sample solution to pass through the opening, and the diameter may be as small as possible. In consideration of sufficient suppression of the evaporation of the sample solution in the cavity, the diameter is desirably ⅓ or less of the diameter of the cavity (in the term of an area, the area of the opening is desirably 1/9 or less of that of the cavity. For example, a projection is formed at the outer circumferential region of the opening 110 since a droplet does not leak from the opening 110 to the outside of the opening 110. A burr formed by cutting or some other method may be used as the projection.

A method for fitting the measurement cavity side wall 15 and the measurement cavity lid 16 air-tightly to each other may be, for example, a method of bonding the two to each other with, for example, an epoxy resin, or a mechanically clamping method. In a case of using silicone for the measurement cavity side wall 15 and glass for the cavity lid, for example, an anodic oxidation method is used to fit the two air-tightly from viewpoint of air tightness. For example, the air-tightly fitting is attained not to cover the filling opening 19 for the sample solution with the measurement cavity lid 16. The measurement cavity side wall 15 and the cavity lid 16 may be integrated with each other.

For example, the sample solution is composed of, in addition to pyrophosphoric acid that is a measurement target, pyrophosphatase, glyceraldehyde-3-phosphate dehydrogenase, diaphorase, glyceraldehyde-3-phosphate, nucleotides, an electron mediator, and a buffer solution component.

From the viewpoint of operation easiness, four reagent layers may be provided, in advance, on the insulating substrate 11 of the sensor inside the measurement cavity 17, where the reagent layers include a first reaction reagent layer containing a buffer solution component, an electron mediator and a magnesium salt, a second reagent layer containing pyrophosphatase, glyceraldehyde-3-phosphate dehydrogenase and diaphorase, a third reaction reagent layer containing glyceraldehyde-3-phosphate, and a fourth reaction reagent layer containing oxidized nicotinamide dinucleotide. It is desirable to divide the components constituting the sample solution into the four reaction reagent layers.

The electron mediator is desirably a water-soluble stable oxidant, and is, for example, potassium ferricyanide. As is understood from Examples that will be described later, as a sample solution in a method for evaluating the detection performance and the detection stability of this sensor, an aqueous solution may be used in which potassium ferricyanide having a molar concentration four times that of pyrophosphoric acid to be measured is dissolved in a solvent such as potassium nitrate. The reason therefor is that one molecule of pyrophosphoric acid which undergoes reaction by aid of the above-mentioned enzyme can change potassium ferricyanide, which functions as the electron mediator, to four molecules of potassium ferrocyanide.

The magnesium salt may be a magnesium salt that contains a magnesium ion, be water soluble, and have a pH varying from neutrality to weak alkalinity. For example, magnesium chloride may be used.

Instead of oxidized nicotinamide dinucleotide (NAD+), oxidized nicotinamide dinucleotide phosphate (NADP+) may be used. In the present specification, oxidized nicotinamide dinucleotide, oxidized nicotinamide dinucleotide phosphate and a combination of the two each refer to as nucleotides.

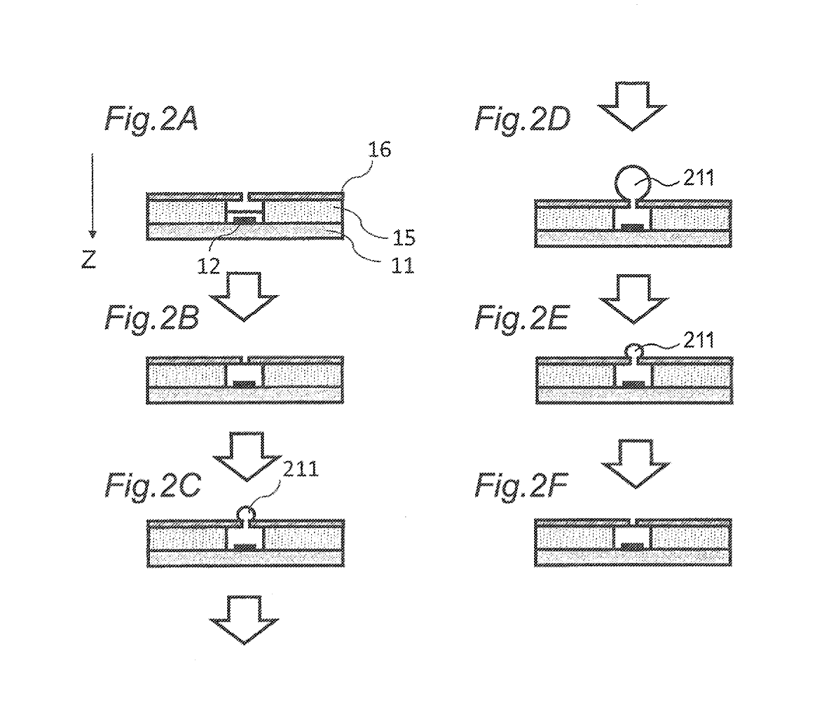

Subsequently, a description will be made about a method for detecting pyrophosphoric acid using the above-mentioned sensor device and the sample solution, referring to FIGS. 2A to 2F.

The sample solution is first supplied from the filling opening 19 through the flow path 18 to the measurement cavity 17 (FIG. 2A). Until the measurement cavity 17 is filled with the sample solution, the supply of the sample solution is continued (FIG. 2B). At this time, it is advisable to use, for example, an external heater at this time to heat the sample solution in the measurement cavity 17, thus promoting a chemical reaction by the enzyme. For example, the sample solution is heated to adjust the temperature of the solution to 30 to 40° C., and the temperature is kept for 5 to 10 minutes.

Thereafter, the sample solution is introduced into the measurement cavity 17 with a volume larger than the volume of the cavity 17, and part of the sample solution is allowed to be exposed from the opening 110 outside the measurement cavity 17 (FIG. 2C). At this time, it is desirable that no volume of a droplet to be exposed seeps outside from the opening 110, and, for example, the droplet does not surpass a limit that the droplet can maintain its shape of the sphere by surface tension. Specifically, the cavity lid may be subjected to a water repellency treatment with HMDS or the outer circumferential region of the opening 110 may be projected, and in order to prevent droplet from seeping to the outside of the opening 110, the volume of the droplet to be exposed may be desirably not more than 0.5 μL. In order to effectively obtain an increase in the detected current by the concentration of the droplet, for example, the volume of the droplet to be exposed is not less than 0.2 μL.

Next, when the sample solution supplied to the measurement cavity 17 is left as it is for a constant period (FIG. 2D), water which is the solvent component of the droplet 211 evaporates (FIG. 2E). The period for leaving the solution depends on the volume of the droplet to be exposed, and the temperature and humidity of the outside. When the droplet has a volume of not more than 0.2 μL, leaving the solution as it is for not shorter than 5 minutes causes the exposed droplet to disappear, thus forming a concentrated layer in the opening 110. At this time, the measurement cavity 17 may be heated with, for example, an external heater to promote the evaporation of the solvent, thus shortening a period for the evaporation. Before and after the droplet 211 is exposed, the temperature of the sample solution may be changed. In order to raise the temperature of the exposed droplet 211, the droplet 211 may be locally heated from the outside by, for example, a laser. Thereafter, the evaporation of the solvent in the droplet ends (FIG. 2F), and subsequently a constant voltage is applied to the measurement electrode 12 to start a measurement of the current. When the droplet has been made small by the evaporation of the solvent, or has not been exposed from the outside of the opening 110, the evaporation speed is remarkably lowered. Thus, the droplet does not to undergo a further concentration. As a result, the measured current is increased and simultaneously variance of the value of the current is reduced. In this case, even if the exposed droplet 211 is returned into the measurement cavity 17 in a state where a slight volume of the droplet 211 remains, substantially the same effect of the current increase based on the concentration can be obtained.

### Example 1

Hereinafter, a more specific description will be made about the detection method of the present disclosure using a biosensor.

A silicon nitride film of a thickness of 100 nm was first deposited onto a silicon substrate of 700 μm thickness as an insulating substrate 11 by plasma CVD. Next, a resist was painted thereon, and photolithography was used to remove the resist on a region where an electrode is formed. Next, electron beam vapor deposition method was used to deposit a titanium thin film of a thickness of 5 nm, as an adhesion layer, thereon and then deposit a gold thin film into a thickness of 100 nm thereon. Thereafter, by lifting-off, unnecessary portions thereof were removed to form a measurement electrode 12, a counter electrode 13, a reference electrode 14 and a terminal. Among the formed measurement electrodes 12, which had various areas, the smallest electrode had an area of 0.49 mm².

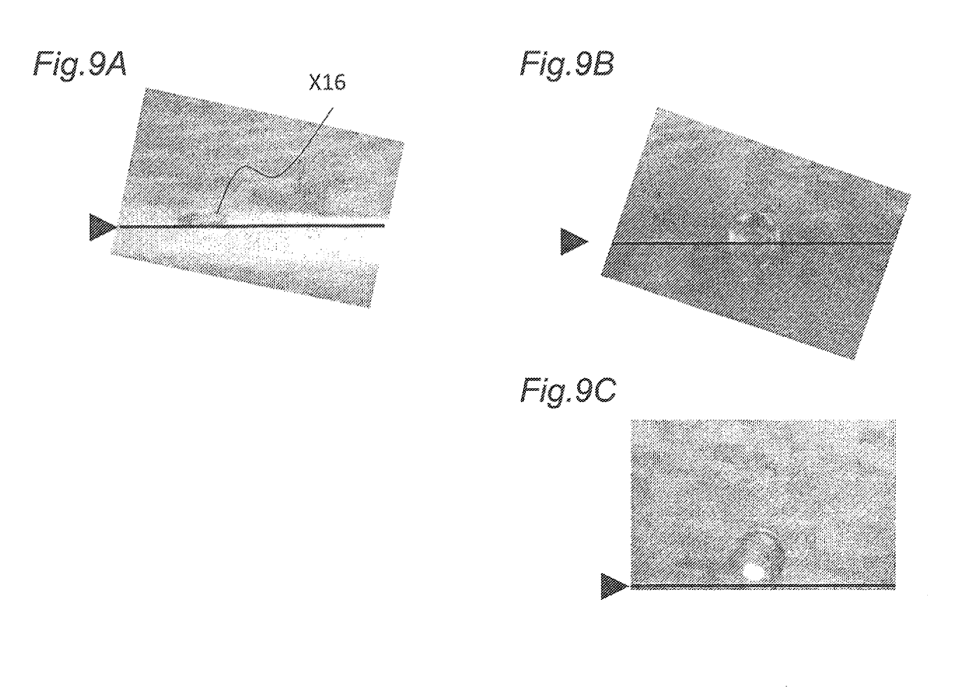

A measurement cavity side wall 15 was formed by pressing a transparent mold made of PMMA onto the substrate in a measurement cavity 17 region, injecting PDMS from the outside of the mold into regions other than the measurement cavity 17, heating/curing the injected PDMS at 85° C. for 30 minutes, and finally removing the mold. The height of the measurement cavity 17, that is, the thickness of the PDMS layer was 230 μm. By this method, a flow path 18 and a filling opening 19 connected to the measurement cavity 17 were simultaneously formed. The cavity region was covered with a PET film having a thickness of 30 μm and having an opening 110 of 600 μm diameter. A surface of the lid was subjected to a water repellency treatment with, for example, HMDS (hexamethyldisilane). The surface corresponds to places shown by triangle marks in FIGS. 9A to 9C. The formed opening 110 had a burr generated by the mechanical work, and thus, the outer circumferential region thereof was projected by about 20 to 40 μm (FIG. 9A). Ultrapure water was dropped onto the PET film subjected to the water repellency treatment, and then the contact angle thereof was measured. As a result, the angle was about 135° (FIG. 9B). When a droplet was exposed from the opening 110, the droplet was restrained from seeping into the surrounding since the outer circumferential region was projected, that is, a convex portion X16 was formed along the outer circumference of the opening. Thus, the contact angle was not less than 150 (FIG. 9C).

The opening 110 in the lid was arranged to be positioned just above the measurement electrode 12. Among the formed measurement cavities, which had various volumes, the smallest measuring cavity had a volume of 0.5 μL. The diameter thereof was 1.8 mm, and the electrode area was 0.49 mm² as described above.

In order to examine the detection sensitivity of this sensor, experiments were conducted under conditions described hereinafter. In view of SNP typing, potassium ferrocyanide was used which has a concentration corresponding to a concentration four times the concentration of 0.1 mM of pyrophosphoric acid generated in a case where a DNA probe undergoes reaction for an elongation of 100 base pairs to be amplified into 500 nM by PCR. By cyclic voltammetry, the redox current was measured between −0.6 V and 0.8 V. With sweeping speed set to 100 mV/second, potassium ferrocyanide was oxidized. The maximum current value at the time of the oxidization was repeatedly measured totally four times.

Sample solution: 0.4 mM solution of potassium nitrate solution containing potassium ferrocyanide
(1) Without measuring cavity lid
(2) With measuring cavity lid
(3) With measuring cavity lid: the sample solution having a volume of 0.2 microliter was exposed from the opening 110, and then concentrated, and subsequently the measurement was made.

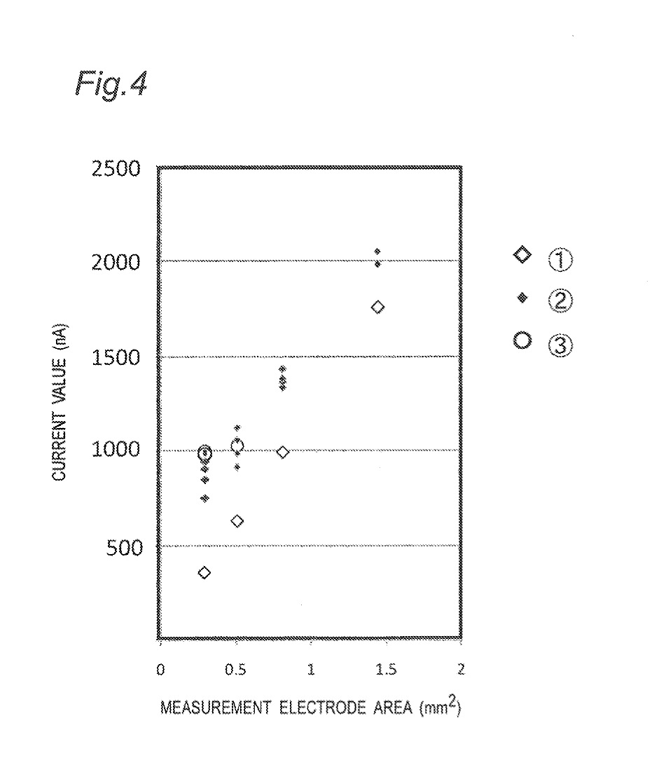

It is desirable that the measured current values are larger and that their measurement variance is smaller. As shown in FIG. 4, under the condition of (1) without measuring cavity lid, the solvent component in the sample solution was evaporated during the measurement, and thus, as the measurement was repeated, the detected current value increased so that the measured values were largely varied (error range: a CV value of about 12%).

Under the condition of (2) with measuring cavity lid, the evaporation was suppressed, and thus, the variance of the measured values was far smaller as compared to the condition (1). However, the detected current value was lowered in proportion to the electrode area.

Under the condition of (3) with measuring cavity lid, the measurement was made after the droplets were concentrated. When the electrode area was small and the volume of the sample solution was small, increase of the current value was observed. The detected current value was at most about 2.2 times that under the condition (2) in which the concentration based on evaporation was not performed. Furthermore, an increase of the current value was not observed, dependently in the number of times of the measurement. A variance of the measured values was small, which was substantially equal to that under the condition (2) (error range: a CV value of 1% or less).

### Embodiment 2

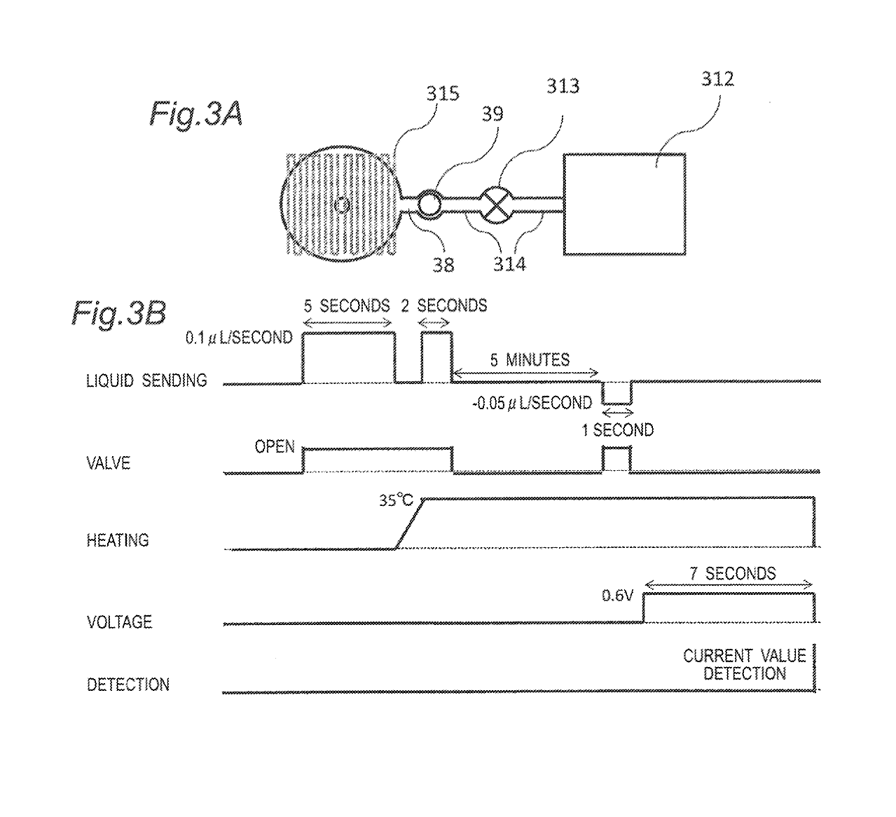

FIGS. 3A and 3B illustrate a configuration of a pyrophosphoric acid sensor and a driving sequence thereof according to one embodiment of the present disclosure. As illustrated in FIG. 3A, in the pyrophosphoric acid sensor in Embodiment 2, a solution sending unit 312 for sending a sample solution and an opening-closing valve 313 for controlling the sending of the solution to a measurement cavity 37 are connected to each other through a capillary 314. The opening-closing valve 313 and a filling part 39 are connected to each other through a flow path 38. Through this system, the sending of the solution to the measurement cavity 37 in the sensor is controlled. Furthermore, just below a sensor substrate, a heater 315 is connected thereto. The configuration of the sensor section is the same as that described in Example 1. Based on the driving sequence illustrated in FIG. 3B, the sample solution is supplied to the sensor section, heated, exposed toward the outside from the cavity, evaporated to be concentrated, and then measured the current value.

### Example 2

As shown in Table 1 described below, 20 μL of a sample solution was formulated. 0.5 μL of the solution was supplied to the measuring cavity 37 of the sensor formed with the configuration of FIG. 3A in Example 1A. Through the driving sequence in FIG. 3B, pyrophosphoric acid was measured. As can be read out from FIG. 3B, the volume of the droplet to be exposed was 0.2 μL.

TABLE 1

| | | | | (Final | |
|---|---|---|---|---|---|
| | | | | concentration) | |
| Measure- | Tricine buffer solution | 1.8 | uL | 45 | mM |
| ment | (pH: 8.8) | | | | |
| solution: | Oxidized nicotinamide | 0.2 | uL | 1 | mM |
| | dinucleotide | | | | |
| | Magnesium chloride | 0.4 | uL | 1.7 | mM |
| | Potassium ferricyanide | 2 | uL | 10 | mM |
| | Glyceraldehyde-3-phosphate | 0.66 | uL | 10 | mM |
| | Diaphorase | 1 | uL | 10 | U/mL |
| | Glyceraldehyde-3-phosphate | 1 | uL | 32 | U/mL |
| | dehydrogenase | | | | |
| | Pyrophosphatase | 0.5 | uL | 5 | U/mL |
| | Water | 10.5 | uL | | — |
| | Pyrophosphoric acid | 2 | uL | 0.3 | mM |

The measurement was made while the thickness of the PDMS layer was varied, which corresponded to the distance between the measurement electrode and the measurement cavity opening. As shown in FIG. 4, by making the thickness of the PDMS layer small, an increase of the detected current value was observed. In a case where the thickness of the PDMS layer 400 μm, the current value increased about 1.3 times larger than that of a case where the thickness was 680 μm or more. Furthermore, in a case where the thickness of the PDMS layer was 230 μm, the current value increased about two times larger than that of the case where the thickness was 680 μm or more. These results suggest that as the concentrated reaction species is nearer to the measurement electrode, the current value is larger, thus showing that the advantageous effects of the present disclosure are more remarkably produced. The measurements were repeatedly made, and as a result, it was found that the variance of the measured values in each measurement were as small as a CV value of 5% or less, which situation is not shown in the graph.

### Embodiment 3

Prepared is a reaction system containing a DNA sample solution, which is an object to be measured by SNP typing, a DNA probe having a sequence complementary to an SNP sequence of the DNA and having an SNP site, DNA polymerase and deoxynucleotide, and then a PCR reaction is caused. When the SNP site of the DNA to be measured by SNP typing was complementary to the DNA probe having the SNP site, the operation described above makes it possible to elongate the DNA probe and further produce pyrophosphoric acid. On the other hand, when the SNP site of the DNA to be measured by SNP typing is not complementary to the DNA probe having the SNP site, the DNA probe is not elongated and pyrophosphoric acid is not produced. Thereafter, the sample solution in which the PCR reaction is ended is mixed with a reaction liquid composed of pyrophosphatase, glyceraldehyde-3-phosphate dehydrogenase, diaphorase, glyceraldehyde-3-phosphate, nucleotides, an electron mediator and a buffer solution component. The mixture is shifted through the flow path 38 to the measurement cavity 37. As a result, pyrophosphoric acid can be quantitatively determined, correspondingly to the type of the SNP, and thus, the SNP typing of the DNA to be measured can be attained. When a reaction system containing DNA polymerase and deoxynucleotide is carried in a PCR reaction cavity, the SNP typing of the DNA to be measured can be attained by injecting, from the filling opening 39, the DNA sample solution, which is an object to be measured by SNP typing. A method for shifting the sample solution from the PCR reaction cavity to the measurement cavity in the sensor may be conducted in the same way as in Example 2.

### Example 3

Hereinafter, a description will be made about an example in which an SNP typing sensor according to one embodiment was used to conduct SNP typing of a DNA in a sample solution.

First, a pyrophosphoric acid sensor was formed in the same way as in Example 1. The measuring method is substantially the same as in Example 2.

A human genome extracted from blood of each of AB blood type and O blood type persons was used as a template for a model of the SNP typing.

The measurement was made from the sixth exon of the human genome, using Control Primer I (5′-TAGGAAGGATTCCTCG-3′: SEQ ID No. 1) and Primer 3 (5′-TTCTTGATGGCAAACACAGTTAAC-3′: SEQ ID No. 2) as primers for amplifying a DNA fragment containing an SNP site, and using Primer 1′ (5′-TAGGAAGGATGTCCTCGTGACG: SEQ ID No. 3), and Primer 3 as a primer for performing SNP typing. This SNP typing primer causes an elongation reaction specifically for AB type blood.

First, 1. 8 μL of template 1 was added to 0.2 μL of KOD-FX polymerase manufactured by Toyobo Co., Ltd., 5 μL of 2×KOD-Buffer, 1 μL of 2 mM dNTP, 1 μL of 10 mM Primer 1, and 1 μL of 10 mM Primer 3 to conduct a PCR reaction in 35 cycles, in each of cases where the temperature was kept at 98° C. for 30 seconds, at 60° C. for 30 seconds and at 68° C. for 30 seconds. Furthermore, the resultant PCR product was diluted 1000 times, and 2 μL of the resultant: was collected as template 2. The collected resultant was mixed with 0.2 μL of Taq-polymerase manufactured by Takara Bio Inc., 3 μL of the PCR mixture, 2 μL of Primer 1′, 2 μL of Primer 3, and 10.8 μL of distilled water and a PCR reaction is conducted in 35 cycles, in each of cases where the temperature was kept at 95° C. for 30 seconds, at 60° C. for 30 seconds and at 72° C. for 30 seconds. From this sample solution, a volume of 10 μL was taken out, and mixed with 2.5 μL of water. Thereafter, this mixture was mixed with a reaction liquid described in Table 2 described below, and then was introduced into the measurement cavity in the sensor in the same way as in Example 2, and the measurement was made. The measurement results are shown in Table 3.

TABLE 2

| | | | | (Final | |
|---|---|---|---|---|---|
| | | | | concentration) | |
| Reaction | Tricine buffer solution | 1.8 | uL | 45 | mM |
| liquid: | (pH: 8.8) | | | | |
| | Oxidized nicotinamide | 0.2 | uL | 1 | mM |
| | dinucleotide | | | | |
| | Magnesium chloride | 0.4 | uL | 1.7 | mM |
| | Potassium ferricyanide | 2 | uL | 10 | mM |
| | Glyceraldehyde-3-phosphate | 0.66 | uL | 10 | mM |
| | Diaphorase | 1 | uL | 10 | U/mL |
| | Glyceraldehyde-3-phosphate | 1 | uL | 32 | U/mL |
| | dehydrogenase | | | | |
| | Pyrophosphatase | 0.5 | uL | 5 | U/mL |

TABLE 3

| | AB type | O type |
|---|---|---|
| | SNP typing | SNP typing |
| Current value after 30 seconds | 198 nA | 48 nA |
| Measurement error: CV value (%) | 7.0% | 9.2% |

As shown in Table 3, the specific elongation reaction was caused specifically for the DNA originating from the AB type blood, and pyrophosphoric acid produced by the specific elongation reaction was detected by the electrochemical measurement in the measurement cavity. On the other hand, for the O type blood, the specific elongation reaction was not caused, and thus the amount of detected pyrophosphoric acid was relatively small, so that a current was hardly detected. The CV value obtained by making the measurement continuously five times was also small. This did not affect the SNP typing. From these results, with a small sensor and a small volume of a specimen a current value of a level at which SNP typing could be attained could be obtained, and simultaneously a stable measurement was realized. Therefore, one base mismatch typing, which is a model of an SNP site, could be attained with a small sensor, using a small amount of a specimen.

One general aspect derived from the above-mentioned embodiments is descried in the followings.

1. A method for detecting pyrophosphoric acid contained in a sample solution, the method including:
(a) preparing a pyrophosphoric acid sensor including
an insulating substrate 11;
a measurement electrode 12 provided on the insulating substrate 11;
a counter electrode 13 provided on the insulating substrate 11;
a measurement cavity side wall 15 provided on the insulating substrate 11; the measurement cavity side wall 15 having a measurement cavity 17 which is a through hole provided in the inside the measurement cavity side wall 15;
a measurement cavity lid 16 which overlaps the measurement cavity 17 when viewed from a normal direction of the insulating substrate 11; and
a flow path 18 which communicates with the measurement cavity 17; wherein
when viewed from the normal direction of the insulating substrate 11 (Z-direction in FIG. 1), the measurement cavity 17 overlaps a portion of the measurement electrode 12 and a portion of the counter electrode 13;
the measurement cavity lid 16 includes an opening 110 which is a through hole,
when viewed from the normal direction of the insulating substrate 11, the opening 110 is included in the measurement cavity 17;
(b) supplying the sample solution having a volume of more than the volume of the measurement cavity 17 to the measurement cavity 17 through the flow path 18, so as to expose a droplet 211 from the opening 110; wherein the droplet 211 has a shape of sphere;
the shape of the sphere is maintained by surface tension generated on a surface of the droplet 211;
(c) evaporating at least part of the sample solution contained in the droplet 211, so as to increase a concentration of pyrophosphoric acid in the sample solution included in the measurement cavity 17;
(d) measuring a current value flowing through the sample solution by using the measurement electrode 12 and the counter electrode 13; and
(e) determining the concentration of the pyrophosphoric acid on the basis of the current value measured in the measuring (d).

This disclosure makes it possible to detect pyrophosphoric acid contained in the sample solution with high sensitivity and high accuracy.

2. The method according to item 1, wherein
the opening 110 is circular;
the measurement cavity 17 is cylindrical; and
a diameter of the measurement cavity 17 is more than three times greater than a diameter of the opening 110.

This disclosure makes it possible to detect pyrophosphoric acid contained in the sample solution with high sensitivity and high accuracy.

3. The method according to item 1, wherein
a projection is formed around the opening 110.

This disclosure makes it possible to detect pyrophosphoric acid contained in the sample solution with high sensitivity and high accuracy.

4. The method according to item 1, wherein
the droplet 211 has a volume of not less than 0.2 microliters and not more than 0.5 microliters.

This disclosure makes it possible to detect pyrophosphonic acid contained in the sample solution with high sensitivity and high accuracy.

5. The method according to item 1, wherein
the measurement cavity 17 has a height of not more than 400 micrometers.

This disclosure makes it possible to detect pyrophosphoric acid contained in the sample solution with high sensitivity and high accuracy.

6. The method according to item 1, wherein
the measurement cavity 17 has a height of not more than 230 micrometers.

This disclosure makes it possible to detect pyrophosphoric acid contained in the sample solution with high sensitivity and high accuracy.

7. The method according to item 1, wherein
the sample solution contains pyrophosphatase, glyceraldehyde-3-phosphate dehydrogenase, diaphorase, glyceraldehyde-3 phosphate, nucleotide, an electronic mediator and a buffer solution component.

This disclosure makes it possible to detect pyrophosphoric acid contained in the sample solution with high sensitivity and high accuracy.

8. The method according to item 1, further including
heating the sample solution, before the evaporating (c).

This disclosure makes it possible to detect pyrophosphoric acid contained in the sample solution with high sensitivity and high accuracy.

9. The method according to item 1, wherein
the sample solution is heated in the evaporating (c).

This disclosure makes it possible to detect pyrophosphoric acid contained in the sample solution with high sensitivity and high accuracy.

10. An SNP typing method using the method according to item 1 is included in the spirit of the present disclosure.

### INDUSTRIAL APPLICABILITY

According to the present disclosure, it is possible to provide a detecting method and an SNP typing method for detecting pyrophosphoric acid in a sample solution with high sensitivity and high accuracy by a small sensor element.

What is claimed is:

1. A method for detecting pyrophosphoric acid contained in a sample solution, the method comprising:
(a) preparing a pyrophosphoric acid sensor comprising:
an insulating substrate;
a measurement electrode provided on the insulating substrate;
a counter electrode provided on the insulating substrate;
a measurement cavity side wall provided on the insulating substrate; the measurement cavity side wall having a measurement cavity which is a through hole provided in the inside the measurement cavity side wall;
a measurement cavity lid which overlaps the measurement cavity when viewed from a normal direction of the insulating substrate; and
a flow path which communicates with the measurement cavity; wherein
when viewed from the normal direction of the insulating substrate, the measurement cavity overlaps a portion of the measurement electrode and a portion of the counter electrode;
the measurement cavity lid comprises an opening which is a through hole,
when viewed from the normal direction of the insulating substrate, the opening is included in the measurement cavity;
(b) supplying the sample solution having a volume of more than the volume of the measurement cavity to the measurement cavity through the flow path, so as to expose a droplet from the opening; wherein
the droplet has a shape of sphere;
the shape of the sphere is maintained by surface tension generated on a surface of the droplet;
(c) evaporating at least part of the sample solution contained in the droplet, so as to increase a concentration of pyrophosphoric acid in the sample solution included in the measurement cavity;
(d) measuring a current value flowing through the sample solution by using the measurement electrode and the counter electrode; and
(e) determining the concentration of pyrophosphoric acid on the basis of the current value measured in the measuring (d).

2. The method according to claim 1, wherein
the opening is circular;
the measurement cavity is cylindrical; and
a diameter of the measurement cavity is more than three times greater than a diameter of the opening.

3. The method according to claim 1, wherein
a projection is formed around the opening.

4. The method according to claim 1, wherein
the droplet has a volume of not less than 0.2 microliters and not more than 0.5 microliters.

5. The method according to claim 1, wherein
the measurement cavity has a height of not more than 400 micrometers.

6. The method according to claim 1, wherein
the measurement cavity has a height of not more than 230 micrometers.

7. The method according to claim 1, wherein
the sample solution contains pyrophosphatase, glyceraldehyde-3-phosphate dehydrogenase, diaphorase, glyceraldehyde-3-phosphate, nucleotide, an electronic mediator and a buffer solution component.

8. The method according to claim 1, further comprising
heating the sample solution, before the evaporating (c).

9. The method according to claim 1, wherein
the sample solution is heated in the evaporating (c).

10. An SNP typing method using the method according to claim 1.


SEQUENCE LISTING (3 sequences)

SEQ ID NO: 1 - DNA, 17 bp, Artificial
  Synthetic construct; Control Primer for amplifying a DNA fragment including an SNP portion
taggaaggatgtcctcg

SEQ ID NO: 2 - DNA, 24 bp, Artificial
  Synthetic construct; Primer for amplifying a DNA fragment including an SNP portion
ttcttgatggcaaacacagttaac

SEQ ID NO: 3 - DNA, 21 bp, Artificial
  Synthetic construct; Primer for conducting an SNP typing
taggaaggatgtcctcgtgac